(19) United States
(12) Patent Application Publication
(10) Pub. No.: US 2013/0101669 A1
(43) Pub. Date: Apr. 25, 2013

(54) LOGICAL ENZYME TRIGGERED (LET) LAYER-BY-LAYER NANOCAPSULES FOR DRUG DELIVERY SYSTEM

(71) Applicants: Mark Appleford, San Antonio, TX (US); Marie-Michelle Kelley, San Antonio, TX (US)

(72) Inventors: Mark Appleford, San Antonio, TX (US); Marie-Michelle Kelley, San Antonio, TX (US)

(21) Appl. No.: 13/623,338

(22) Filed: Sep. 20, 2012

Related U.S. Application Data
(60) Provisional application No. 61/538,086, filed on Sep. 22, 2011.

(51) Int. Cl. A61K 9/51

(52) U.S. Cl. CPC A61K 9/5138; A61K 9/5115; Y10S 977/773; Y10S 977/906; B82Y 5/00
USPC 424/463; 514/15.2; 977/773; 977/906

(57) ABSTRACT

Nanocapsule compositions comprising a calcium carbonate core surrounded by a bilayer or bilayers of polystyrene sulfonate and poly(allylamine hydrochloride). The poly(allylamine hydrochloride) is conjugate to a substrate, wherein the substrate is capable of being acted upon (for example cleaved) by a biomarker or enzyme associated with a disease state of interest. The nanocapsule compositions may be administered to an animal, for example a human, for the treatment of a disease state.

### STATEMENT OF RIGHTS TO INVENTIONS MADE UNDER FEDERALLY SPONSORED RESEARCH

This invention was made in part during work supported by a grant from the UTSA MBRS-RISE PROGRAM. Grant Number GM60655 with Edwin J Barea-Rodriguez as the Principal Investigator. The government may have certain rights in the invention.

### FIELD OF THE INVENTION

The present invention relates generally to drug delivery systems, and more specifically to the use of layered nanocapsules for the local delivery of therapeutic agents.

### BACKGROUND

### Nanotechnologies and Cancer Therapeutics

Breast cancer is the second leading cause of morbidity and mortality among women in the United States. Early detection and treatment methods have resulted in 100% 5-year survival rates for stage 0-I breast cancer. Unfortunately the 5-year survival rate of metastatic breast cancer (stage IV) is reduced fivefold [1]. The most challenging issues of metastatic breast cancer treatment are the ability to selectively target the adenoma and adenocarcinoma cells both in their location of origin and as they metastasize following initial treatment. Multilayer/layer-by-layer (LbL) nanocapsules have garnered vast interest as anticancer drug delivery systems due to their ability to be easily modified, their capacity to encapsulate a wide range of chemicals and proteins, and their improved pharmacokinetics [2]. Multilayer nanocapsule formation involves the layering of opposing charged polyelectrolytic polymers over a removable core nanoparticle.

Nanoparticles currently command a market in excess of $5.4 billion per year where breast cancer is the field of medicine with the greatest presence of nanotechnological therapeutic agents in the clinic [5, 17]. This major presence may be attributed to nano-therapeutic properties that include biocompatibility, low toxicity, lower clearance rates, the ability to target specific tissues and controlled release of drugs [18]. It has been shown that even in ideal cases, between 1 and 10 parts per 100.000 of intravenously administered monoclonal antibodies reach their parenchymal targets in vivo [19, 20]. Several nanotechnological approaches have been used to improve delivery of chemotherapeutic agents to cancer cells with the goal of minimizing toxic effects on healthy tissues while maintaining antitumor efficacy [15]. Specifically, nanoparticle albumin-bound (nab) paclitaxel have been developed as an attempt to reduce the toxicity of taxanes administration and improve antitumor efficacy. Abraxane, brand name for nab paclitaxel, has been shown to allow for shorter infusion times (30 minutes vs. 3 hours) and less incidence of peripheral neuropathy for patients [21, 22].

### Albumin

As shown with the use of nab-paclitaxel for metastatic breast cancer (MBC) in the clinical setting, albumin is emerging as a versatile protein carrier for drug targeting and for improving the pharmacokinetic profile of drugs [23]. Human albumin (66.5 KDa) is a multifunctional, negatively charged plasma protein. Albumin is the most abundant protein in human plasma (50%), where two thirds of total body content is in the extravascular compartment and is a biological therapeutic: it is typically used for treating shock, burns, trauma, and acute respiratory distress [23-25]. There is great interest to exploit the carrier properties for the development of novel therapeutic reagents for drug delivery. Specifically, the center of the molecule is made up of hydrophobic radicals which are binding sites for many ligands, while the outer part of the molecule is composed of hydrophilic ligands [25].

### Layer-by-Layer (LbL) Nanocapsules

Microencapsulation is a promising technique for biomedical applications [26]. In the field of drug delivery, there is an urgent need for temporal and spatial controlled drug delivery systems. Specifically, the primary focus is the development of intelligent carriers for therapeutic molecules where such therapeutics depend on suitable carriers to protect them from extracellular enzymes and to deliver them to the target cells. The Layer-by-Layer technique was first introduced in the early nineties by Gero Decher and was first applied to charged planar substrates. The technique was later extended to colloidal substrates by 1998 [27]. The adsorption of the polymer onto the colloidal substrates, to form capsule, occurs in the same manner as planar substrates: consecutive deposition of complementary polymers onto colloidal substrate. However, capsule formation is followed by the removal of the sacrificial colloidal substrate (core) [28, 29]. It is therefore noted, that the two fundamental components for capsule fabrication are the core templates and the polyelectrolyte pair [30]. LbL nanocapsules have undergone a remarkable evolution to become a promising drug delivery system [31]. Their attractiveness as drug delivery systems can be attributed to the following properties: size, composition, porosity, stability, surface functionality, colloidal stability, the absence of hazardous procedures, and the use of simple building blocks [27, 28].

### Calcium Carbonate

The sacrificial core, which is a fundamental component of nanocapsule, for our application is calcium carbonate. Calcium carbonate is a naturally occurring mineral with great biocompatibility, and has been proven to intensify enzyme performance [32-34]. As a biological material, calcium carbonate has unique structures and morphologies: calcite (rhomboeder), aragonite (needles), and vaterite (polycrystalline spheres). Where, calcite is a thermodynamically stable form and the remaining forms are metastable [35, 36]. It has been proven that surfactants can influence nucleation, crystal growth and aggregation where the surfactant is used as microreactors for preparation of specific morphologies and sizes [37]. Furthermore calcium carbonate has been widely used in technology, medicine, and microcapsule fabrication [33]. In terms of microcapsule fabrication, calcium carbonate microparticles have proven to be excellent sacrificial templates not only for the fabrication of hollow polyelectrolyte capsules, but also for making “filled” polyelectrolyte capsules since calcium carbonate microparticles can be easily loaded with macromolecules during (co-precipitation method) or after (direct physical adsorption) their preparation [27].

### Polyelectrolytes

In the field of life sciences, applications of polyelectrolytic capsules are ranging from drug delivery, targeted gene therapy, molecular sensing, vaccination, and to biosensor devices [38]. Capsule wall composition plays a crucial role in the fabrication of functional polyelectrolytic capsules, as their porosity strongly depends on the molecular weight and chemical structure of the polyelectrolyte pairs used [30]. Capsule wall composition is based on the electrostatic attraction between oppositely polyelectrolytes (charged polymers) where alternating adsorption of anionic and cationic polyelectrolytes lead to capsule wall formation [39]. Examples of cationic polyelectrolytes are polyvinyl-ammonium chloride and poly-4-vinyl-N-methyl-pyridinium bromide. Examples of anionic polyelectrolytes are potassium polyacrylate, polyvinylsulfonic acid, and sodium polyphosphate [40]. A typical polyelectrolyte capsule described in literature are composed of pairs of synthetic anionic poly(sodium) styrene sulfonate (PSS) and cationic poly(allylamine) (PAH) hydrochloride[30]. These PSS/PAH bilayer nanocapsules are known to be reproducible, do not suffer from capsule aggregation or capsule decomposition upon removal of the core template, and are non-degradable[27].

### Matrix Metalloproteinases (MMP)

Extracellular matrix (ECM) macromolecules, such as matrix metalloproteinases (MMPs), are important for creating the cellular environments required during development and morphogenesis. MMPs are a family of over 20 enzymes that are characterized by their ability to degrade the extracellular matrix (ECM) and their dependence upon Zn²⁺ binding for proteolytic activity [41]. Their targets include other proteinases, proteinase inhibitors, clotting factors, chemotactic molecules, cell surface receptors, cell-cell adhesion molecules, and virtually all structural extracellular matrix proteins. Members of MMP gene family are often grouped according to their modular domain structure: collagenase, stomelysins, and gelatinase. All MMPs have an N-terminal signal sequence (pre domain) that is removed utter it directs their synthesis to the endoplasmic reticulum. MMP-2 and MMP-9 are considered a subclass of the MMPs due to the gelatinolytic activity and have been shown to participate in the wound healing response, and are abundantly expressed in various malignant tumors [42, 43]. These enzymes, gelatinase-A (MMP-2) and gelatinase-B (MMP-9), also have three repeats of a type II fibronectin domain inserted in the catalytic domain, which bind to gelatin, collagens, and laminin [44-46].

ECM degradation is precisely regulated under normal physiological conditions [46]. In normal tissue, homeostasis is established between MMPs and their inhibitors maintaining a proteolytic balance. However, during cancer progression the balance is disturbed resulting in MMP overexpression [47]. Tumor invasion, metastasis, and angiogenesis require controlled degradation of ECM, and increased expression of matrix metalloproteinases (MMPs) [44]. Hence, MMPs are upregulated in malignant disease, and this correlates with advanced tumor stages, increased invasion, metastasis, and shortened survival [45, 48]. It has been shown that both MMP-2 and MMP-9 play an important role in breast cancer and can serve as prognostic biomarkers for breast cancer [42, 49, 50].

### Biological Finite State Machines

Digital systems work with discreet quantities, which can be designed so that for a given input, there is an exact output [51]. Devices that convert information from on form into another according to a definite set of procedures are known as automata [52]. It has been demonstrated that autonomous programmable computers can be created by using biological molecules as input data and biologically active molecules as outputs: thereby producing a system for ‘logical’ control of biological processes [53]. The response of a molecule to stimulation is a common phenomenon [54] and can be extended within a biological system. Therefore, the promise of computers made from biological molecules lies in their potential to operate within a living organism, act autonomously, process the preprogrammed medical knowledge, and output a therapeutic drug [55].

### SUMMARY

The present invention relates to the use of layered nanocapsules for the local delivery of therapeutic agents. In one embodiment, the nanocapsules of the present invention comprise a calcium carbonate core surrounded by a bilayer or bilayers. The bilayer comprises polystyrene sulfonate and poly(allylamine hydrochloride), and the bilayer substantially surrounds the calcium carbonate core. The poly(allylamine hydrochloride) is conjugate to a substrate, wherein the substrate is capable of being acted upon (for example cleaved) by a biomarker or enzyme associated with a disease state of interest.

In one embodiment, the nanocapsules may be administered to an animal, for example a human, for the treatment of a disease state. The substrate to be used will be determined by the disease state to be treated, and the substrate will be acted upon by a biomarker or enzyme associated with the disease state to be treated.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows a precipitation reaction between calcium carbonate and sodium carbonate with polystyrene sulfonate schematic showing calcium carbonate nanoparticles fabrication.

FIG. 2 shows: (A) Physical adsorption schematic shows CaCO3 particles incubating in BSA-FITC solution and BSA-FITC adhering to CaCO3 surface. (B) Co-precipitation schematic showing BSA-FITC conjugation added to CaCl2 solution before mixing with Na2CO3+PSS solution.

FIG. 3 shows a LET nanocapsule schematic: (A) LET nanocapsule with protected paclitaxel, before MMP-9 cleaving of substrate, and mmp-9 mediated degradation; (B) LET nanocapsule with protected paclitaxel, before MMP-2 cleaving of substrate, and MMP-2 mediated degradation; (C) Logic enzyme triggered release of paclitaxel. Note PSS layer is not represented in this schematic.

FIG. 4 shows: (A) Two input and gate with MMP enzyme inputs and chemotherapeutic, paclitaxel, release; (B) LET nanocapsule truth table showing release of paclitaxel only when both MMP-2 and MMP-9 are present.

FIG. 5 shows: (A) Size distribution curve demonstrates that CNTs mean diameter is 315.9±1.4 nm, (n=150) and also indicate that nano-templates are mono-dispersed: (B) Zeta potential shows nano-template zeta potential equal −15.28±01 mV (n=150) and are negatively charged. Zeta potential is used to predict the long-term stability of nanoparticles where there is a direct correlation between the absolute value of zeta potential and template stability.

FIG. 6 shows: (A) SEM image (1 um scale) of nano-template which confirms uniformity of particle size and shape. (B) SEM of (200 nm scale) same batch of calcium carbonate nanoparticle.

FIG. 7 shows a calcium carbonate nanoparticle FTIR spectrum, peaks observed at 800 cm-1 and 1400 cm-1 demonstrate carbonate ion present in template.

FIG. 8 shows: (A) Measured absorbance of BSA-FITC conjugation at 280 nm and 495 nm wavelengths. FITC/BSA molar ratio=2.024, n=3, indicating efficient BSA-FITC conjugation (n=3, SD). (B) Measured fluorescent intensity of BSA-FITC conjugation illustrating linearity of BSA concentration and fluorescence (n=3, SD).

FIG. 9 shows fluorescent intensity of BSA loaded calcium carbonate nanoparticles that were incubated for different times: 1 hr, 2 hrs, 6 hrs, 12 hrs. 18 hrs, 24 hrs, and 36 hrs. Graph indicates that 24 hrs or 36 hrs incubation times exhibited significantly greater fluorescent intensities when compared to incubation times less than 24 hrs (n=3, p<0.001, SEM).

FIG. 10 shows: (A) BSA-F concentration vs. supernatant fluorescent intensity graph demonstrates that BSA encapsulation via co-precipitation is significantly efficient than physical adsorption (* p<0.0001, n=5, SEM): (B) BSA encapsulation efficiency (%) for both methods vs. BSA concentration graph indicates that encapsulation efficiency of co-precipitation method is significantly greater than direct physical adsorption method. Maximum encapsulation efficiency (97.5%) was seen at BSA-FITC concentration of 0.50 mg/mL.

FIG. 11 shows FTIR spectrums of calcium nanoparticles loaded with bovine scrum albumin where BSA amide I region is observed at 1500-1550 cm-1 and carbonate ion peaks observed at 800 cm-1 and 1400 cm-1 demonstrate conserved; (A) loaded with BSA-FITC concentration ranging from 0 ug/mL to 100 ug/Ml; (B) loaded with 0 ug/mL BSA-FITC concentration (blue) and with 100 ug/mL BSA concentration (red) demonstrating absence of BSA in 0 ug/mL spectrum and confirming BSA loading in 100 ug/mL spectrum.

FIG. 12 shows SEM images of calcium carbonate nanoparticles (A) without PSS (B) with 10 mg/mL PSS demonstrating a strong correlation between PSS and calcium carbonate nanoparticle size (C) without PSS where SEM image taken after 24 hrs of re-suspension, indicating that PSS may play a role in the stability calcium carbonate nanoparticles morphology. (D) with PSS where SEM image was taken after 30 days of re-suspension, indicating that PSS may play a role in the stability of calcium carbonate nanoparticle morphology.

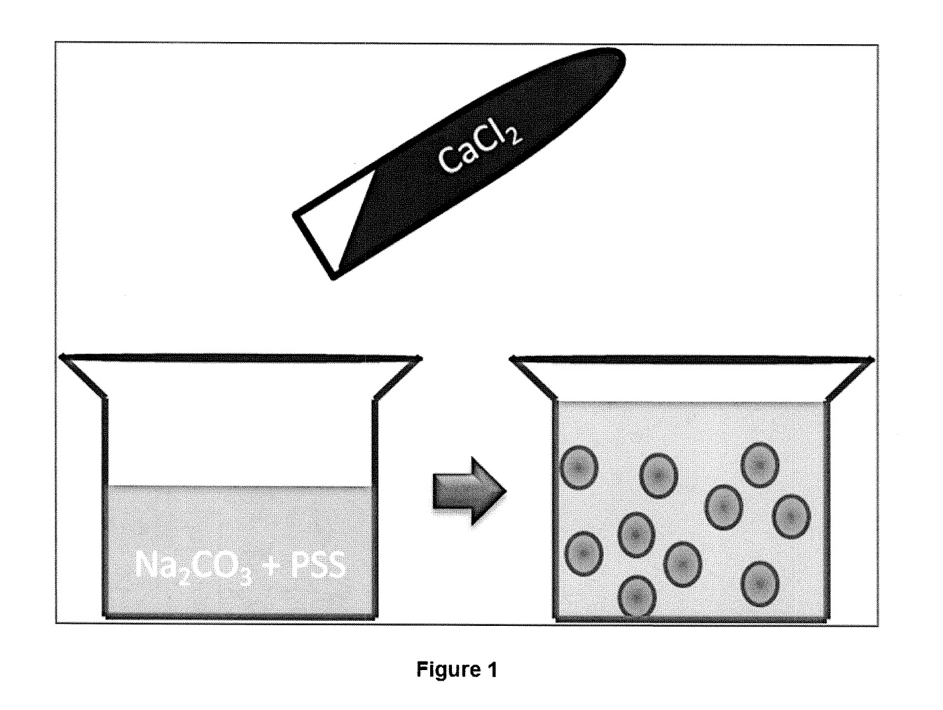

### DETAILED DESCRIPTION OF PREFERRED EMBODIMENTS

The present invention relates generally to drug delivery systems, and more specifically to the use of layered nanocapsules for the local delivery of therapeutic agents.

In one embodiment, the present invention comprises nanocapsules which degrade only after contacting specific biomarkers associated with a given disease state. The nanocapsules are fabricated using layer-by-layer (LbL) technology coupled with extracellular matrix (ECM) protein substrates, which results in an enzyme triggered LbL nanocapsule drug delivery system.

In another embodiment, the nanocapsules comprise a calcium carbonate core surrounded by a bilayer. The bilayer comprises polystyrene sulfonate and poly(allylamine hydrochloride), and the bilayer substantially surrounds the calcium carbonate core. The bilayer may comprise several sub-bilayers, with the number of sub-bilayers ranging from approximately 1 to 10, for example 3-7. The poly(allylamine hydrochloride) is conjugate to a substrate, wherein the substrate is capable of being acted upon (for example cleaved) by a biomarker or enzyme associated with a disease state of interest.

In one embodiment, the nanocapsules may be administered to an animal, for example a human, for the treatment of a disease state. The substrate to be used will be determined by the disease state to be treated, and the substrate will be acted upon by a biomarker or enzyme associated with the disease state to be treated. For example, the disease state to be treated may be breast cancer, and the substrate may be an MMP-cleavable substrate capable of being cleaved by MMP present in breast cancer cells. In this example, the MMP may be MMP-2 and/or MMP-9. In some embodiments, the nanocapsules may comprise two or more substrates, wherein each substrate is capable of being acted upon by a different biomarker for the disease state. For example, nanocapsules for the treatment of breast cancer may include substrates capable of being acted upon by MMP-2 and MMP-9, and the degradation of the nanocapsule may only be accomplished when both MMP-2 and MMP-9 are present.

### Example 1

For the logical enzyme triggered (LET) nanocapsules to be successful as therapeutic delivery vehicles, three areas must be addressed: (i) encapsulation of the therapeutic, (ii) release of the therapeutic and, (iii) targeting in biological systems [28]. These three pertinent areas of the invention have been investigated for the following.

1. Calcium carbonate nanoparticles

2. LbL nanocapsules with calcium carbonate nano-core

3. LET nanocapsules

### Calcium Carbonate Nanoparticles

The role of the calcium carbonate nanoparticles in the making of LbL nanocapsules is twofold: temporary foundation for LbL nanocapsule synthesis and vehicle for therapeutic drug encapsulation. Multilayer nanocapsule formation involves the layering of polyelectrolytes on a sacrificial core which is a fundamental component to LbL nanocapsule synthesis [1, 2]. Various substrates have been used as sacrificial cores: silica, melamine formaldehyde and polystyrene beads silica nano-templates are conventionally used for LbL nanocapsule formation. These substrates offer the following advantages: water solubility, efficient conjugation, and low cytotoxicity [56].

Silica core synthesis can take up several days [26, 57, 58] and core removal requires the use of an extremely corrosive and difficult to handle solvent, hydrofluoric acid [30]. Melamine formaldehyde nanoparticles (MF), although conventionally used, have their own disadvantages. Removal of MF-cores is more difficult as they stay to the capsule wall and/or in the capsule interior [27]. In contrast, calcium carbonate microparticles are nontoxic and can be dissolved by ethylene diamine tetraacetic acid [30, 59]. The major advantage of calcium carbonate cores is the low molecular weight of the ions [27].

Reproducible fabrication of calcium carbonate nanoparticles has not been established. However, there are various methods for calcium carbonate (CaCo₃) micro-particle fabrication such as micro-emulsion, high-aravity reactive and precipitation [36, 60, 61]. Of the various methods precipitation is the simplest and most cost efficient. It has been shown that sodium carbonate mixed with calcium chloride yielded consistent homogeneous spherical microparticles compared to ammonium bicarbonate mixed with calcium chloride [62]. Therefore, a simple and reproducible fabrication method for CaCO₃ nanoparticles is disclosed herein. One of skill in the art would understand that various alternative methods are also suitable, as described above.

For any method of nanoparticle drug incorporation, free unbound drug is leftover in the supernatant during drug loading process. Chemotherapy drugs are relatively expensive, and therefore it is imperative to use a loading method which yields the least amount of drug (waste) in supernatant thereby minimizing the amount of discarded drug. Human protein bovine albumin serum (BSA) has been established in literature as a substitute for actual chemotherapeutics [26, 62, 63]. Therefore in this example, BSA conjugated with fluorescein isothiocyanate (FITC) is incorporated into calcium carbonate nanoparticles using two methods for drug loading: direct physical adsorption and co-precipitation. The efficacy of the two methods is evaluated by calculating the encapsulation efficiency and loading capacity. Spectroscopy is used to measure fluorescent intensities of BSA-FITC, where both BSA encapsulation efficiency (EE) and loading capacity (LC) percentages are calculated using formulas shown below.

$$\begin{matrix}Encapsulation efficiency formula \\ EE \% = \frac{total protein - free protein}{total protein} \times 100 \% & Equations 1 \\ Loading capacity formula \\ LC \% = \frac{total protein - free protein}{nanocapsules weight} \times 100 \% & Equations 2\end{matrix}$$

In drug delivery systems, the targeted drug can be delivered either inside or outside the cell [31]. However having the ability to control calcium carbonate nanoparticle size can expand the LLT LbL nanocapsules to more applications. Wei et al, have investigated effects of anionic surfactants (sodium dodecylsulfonate, sodium dodecylbenzenesulfonate and poly(N-vinyl-1-pyrrolidone)) and have found that CaCO₃ morphology is dependent on the anionic surfactant [37]. Polystyrene sulfonate is also an anionic surfactant but its role in CaCO₃ nanoparticle's mean diameter and stability is not fully understood. Cai et al. hake shown PSS to control calcium carbonate nanoparticle size but, the fabrication method differs from this project. It was not known if polystyrene, in conjunction with precipitation reaction between calcium chloride and sodium carbonate, has the same effect as seen in Cai's group [64]. Therefore CaCO₃ nanoparticles have been characterized in terms of mean diameter and zeta potential as the amount of polystyrene added to precipitation reaction is changed during nanoparticle synthesis.

Finally, targeting of the nanoparticles in biological systems has been investigated. There are two objectives: to ascertain the calcium carbonate nanoparticle mean diameter where they remain in the extracellular space and the calcium carbonate nanoparticle biocompatibility.

The effect of nanoparticles associated with human exposure has not been well studied [65]. A common technique seen in literature is to incubate the nanoparticles with cells of interest and perform MTT (dead/alive) assay to determine nanoparticle cytotoxicity [26, 57, 66]. Cell death can be attributed to several morphological or distinct biochemical pathways [67]. However, apoptosis is a type of cell death that is accomplished by specialized cellular machinery [68]. Kroemer et al. asserts that the measurement of DNA fragmentation or of caspase activation may be helpful not only in diagnosing apoptosis, but also in defining the type of cell death, that is, apoptosis associated with caspase activation [67]. Therefore, Caspase 3 and 7 activities in HMEC and MCF-7 cell line-incubated with nanoparticles-will be measured using a Caspase-Glo assay kit (Promega). Followed by, the use of Quant-iT PicoGreen dsDNA reagent to quantify double-stranded DNA (dsDNA) per day.

### Example 2

### LbL Nanocapsule

LbL nanocapsules were designed, fabricated, and characterized, using calcium carbonate nano-core. Calcium carbonate micro-cores have been layered with polystyrene sulfonate (PSS) and poly(allylamine hydrochloride) (PAH) to create LbL microcapsules [69-71]. Shu et al. produced multilayer nanocapsules using silica nano-cores confirming the feasibility of creating nanocapsules. However the LbL nanocapsules were prepared via layer-by-layer assembly of water-soluble chitosan and dextran sulfate.

In addition, multilayer nano-assemblies using PSS/PAH been seen using calcium phosphate or melamine formamide nanoparticles as sacrificial cores [73, 74]. In the presently disclosed embodiment, calcium nanoparticles were synthesized as nano-cores to create LbL nanocapsules.

### Example 3

### LET Nanocapsule

LET nanocapsule efficacy is evaluated based on the following criteria: (i) encapsulation of the therapeutic LET nanocapsule, (ii) release of the therapeutic from LET nanocapsules and, (iii) anticancer targeting of LET nanocapsule in biological system.

Nanocapsules have attracted vast interest for drug delivery applications. There have been attempts at rendering these capsules “smart” where cargo release is dependent on capsule stimulus: pH, temperature, and light. This approach has been successful, but one problem remains: there are no safeguards. In other words, there is no check and balance system to evaluate or validate the stimulus. A solution is the addition of Boolean logic to nanocapsule structures which would produce a ‘logically’ controlled drug delivery system. Biomolecular computer technology will allow the use of biological molecules as input data and biological active molecules as output [53]. Maltzahn et al. have demonstrated the feasibility of building logical AND/OR gates by conjugating ECM enzymes with nanoparticles [75]. In addition another group has developed the release of liposomes' content mediated by ECM enzyme [76].

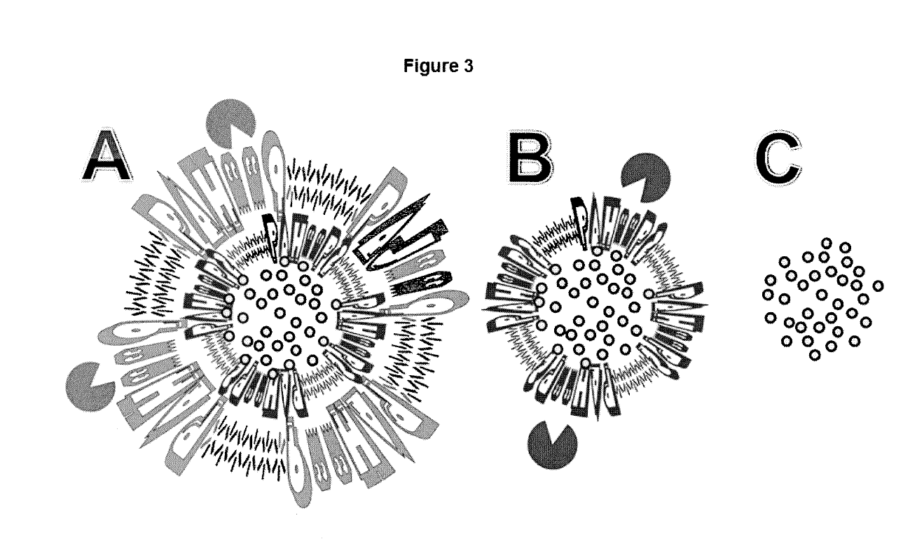

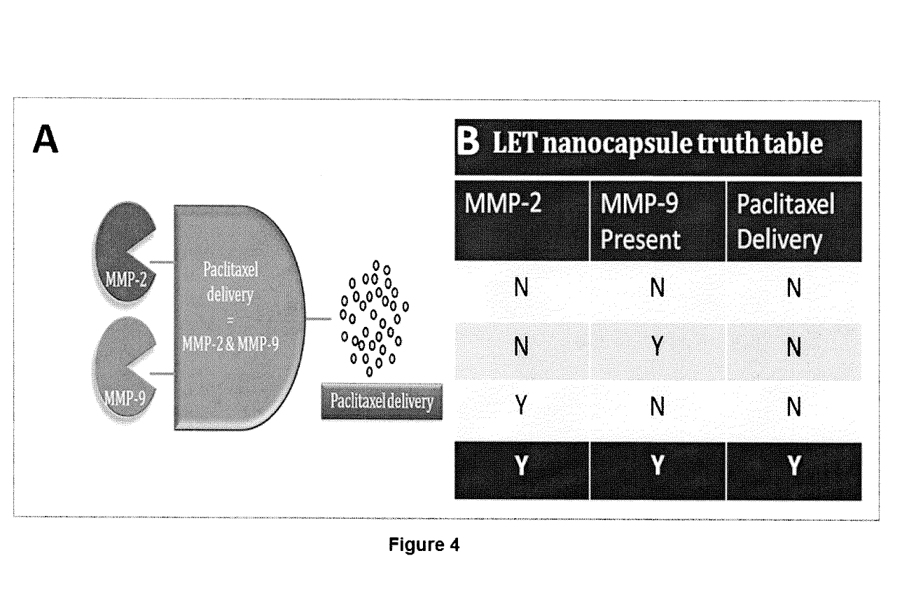

It has been shown that from approximately eight layers (4 bi-layers) onwards the permeability is controlled by the thickness increase, where the diffusion-limiting region is the polyelectrolyte layer [77]. In this embodiment, 5 bi-layers were deposited on the CaCO₃ nano-cores. In a recent study, MMP-2 was able to recognize a cleavable peptide sequence (GPLG↓VRGK) and ignore a scrambled control peptide (GVRL↓GPGK) (wherein a downward arrow indicates the point of cleavage). Although the latter control probe has a GVR leader sequence, there was cleavage of the former but not the latter control probe [78]. It has been demonstrated that consensus peptide (VPLS↓LVSG) is the best substrate tested for MMP-9 enzyme [79]. Furthermore, it has been shown that cells can interact with peptides incorporated in polyelectrolyte multilayer films [80]. A simple and versatile method based on electrostatic attraction between oppositely charged polyelectrolytes has shown the potential for peptide immobilization. This simple peptide-polyelectrolyte deposition technique possesses several advantages for peptide conjugation to biomaterials: many polyelectrolytes possess functional groups that can conjugate peptides and the electrostatic attraction between multiple oppositely charged polyelectrolytes provides “covalent bond-like” interactions to prevent immobilized peptide desorption [81]. Therefore, the present disclosure describes enhancement LbL technology using both MMP-2/MMP-9 cleavable substrates where they will be covalently bounded to the PAH (that possesses an amino functional group) and embedded them into the multilayer architecture (Figure: A-C) to implement a two input logical “and” drug delivery system (FIG. 3).

A 16-amino acid oligopeptide containing MMP cleavage substrate with cysteine residues at opposite ends is used as a crosslinking oligomer. The MMP-2 oligopeptide sequence is Ac-GCRDGPLG↓VRGKDRCG-NH, and the MMP-9 oligopeptide sequence is Ac-GCRDVPLS↓LVSGDRCG-NH₂. The control crosslinking oligomer is not cleaved by the enzymatic actions of MMPs. The oligopeptide-PAH conjugation will begin with the grafting of maleimide groups to the PAH sidechains with a coupling reaction between the thiol groups of cysteine (C) and the maleimide groups [81, 82].

The present disclosure describes using ECM molecules as logic gates by forming layer by layer of ECM protein substrates. The over-expression of protein signals (MMP-2 and MMP-9) in breast cancer is documented and can serve as examples of selective, tissue specific signals for a targeted release of anticancer therapies via nano-delivery platforms. A layer by layer enzyme mediated system will be immobilized onto the surface of a sacrificial nano-shell template to selectively ‘open’ in response to extracellular breast cancer signals. The rationale for the process follows an authenticated pathway for the platform. As the outer layer of the platform encounters the proteins secreted by cancer cells, the layer activates the corresponding enzyme to cleave and reveal the next layer in the platform. This on-off signal ensures that the encapsulated drug is not released from the nanoparticle until at least two chemical checkpoints are reached allowing for substantiated drug release.

### Example 4

### Calcium Carbonate Nanoparticle Fabrication and Characterization

Calcium carbonate nanoparticle (CCN) fabrication was carried out as follows. The calcium carbonate nanoparticles were constructed by adapting previously detailed literature reports [37, 60, 64, 69, 70, 83]. Mono-dispersed CCNs were fabricated by a precipitation reaction between sodium carbonate (0.005 mol, 30 mL) and calcium chloride (0.005 mol. 30 mL) under rigorous stirring. Polystyrene sulfonate (PSS) was added to NaCO₃ solution to decrease CCN size and poly-dispersity [60, 64, 71]. The particles were then retrieved by centrifugation and washed with deionized water.

Calcium carbonate nanoparticle were characterized as follows. Calcium carbonate nanoparticle's mean diameter, distribution, stability, and surface charge were measured by submicron particle analyzer. The morphologies of CCNs were further characterized by SEM. Lastly, FTIR was used for chemical analysis of CCNs.

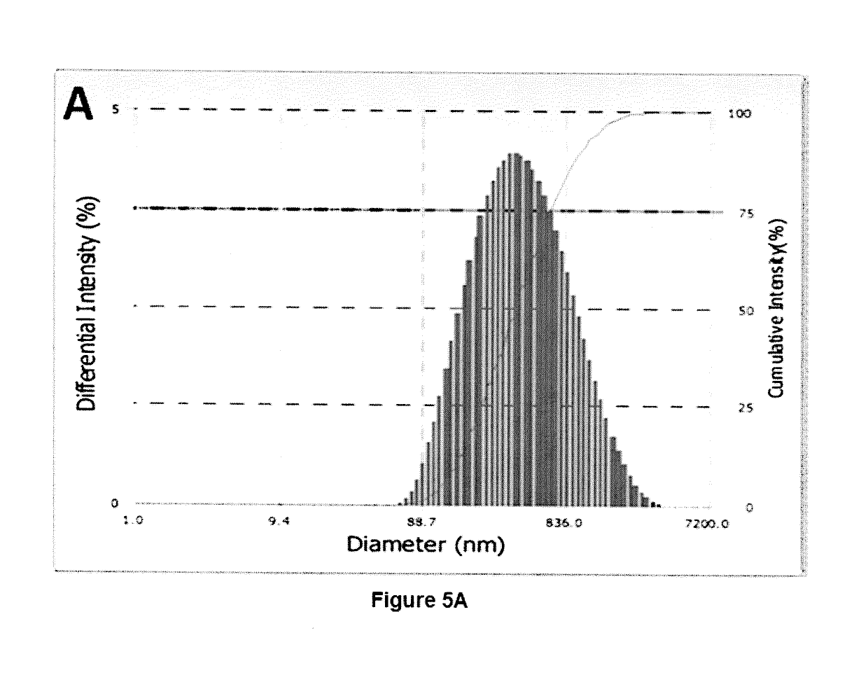

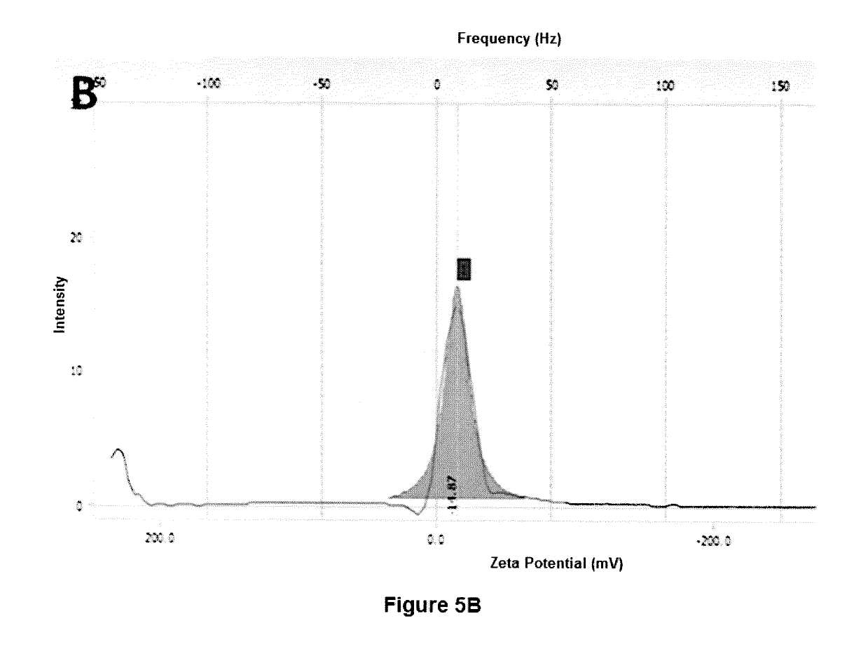

Calcium carbonate nanoparticle mean diameter, distribution and surface charge were characterized as follows. Particle size, distribution, stability, and surface charge were measured by submicron particle analyzer (Delsa Nano). Calcium carbonate nanoparticle sample preparation involved re-suspending (1 mg/mL) de-ionized water, sonicating and ending with vortexing. Mean diameter (FIG. 5A) was measured as 315.9±1.4 nm. Zeta potential is used to predict the long-term stability of nanoparticles where there is a direct correlation between the absolute value of zeta potential and template stability. Zeta potential of the calcium carbonate nanoparticles FIG. 5B) was found to be −15.28±01 mV indicating a stable template with negative surface charge.

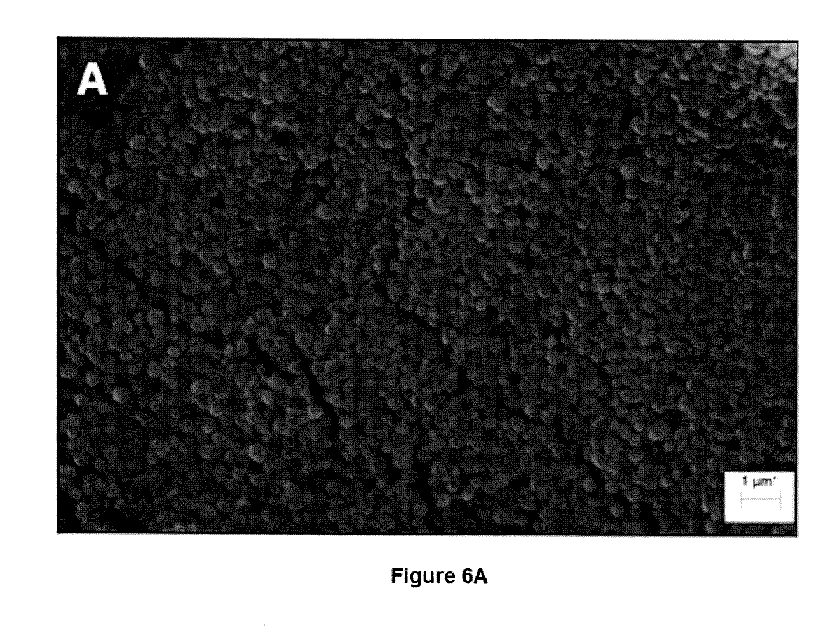

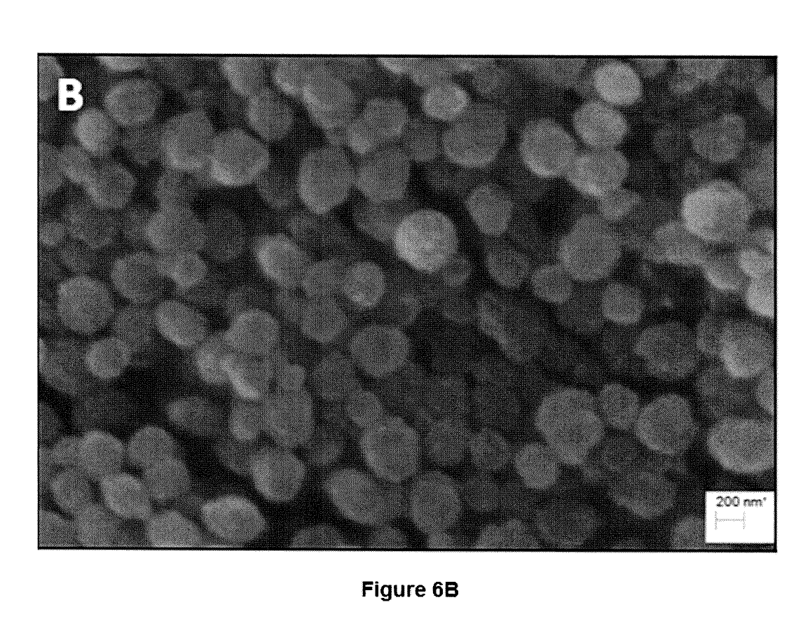

The morphology of calcium carbonate nanoparticles was characterized using scanning electron microscopy (SEM). Calcium carbonate nanoparticle sample preparation involved re-suspending (1 mg/mL de-ionized water, sonicating and ending with vortexing. A 1.5 uL drop of suspension was placed on SEM 9 mm carbon tab and to dry under a hood. The samples were sputtered coated with gold/palladium and imaged using Ziess EVO40 SEM. Calcium carbonate nanoparticles (FIG. 6: A, B) were found to be spherical, rough, and non-aggregated.

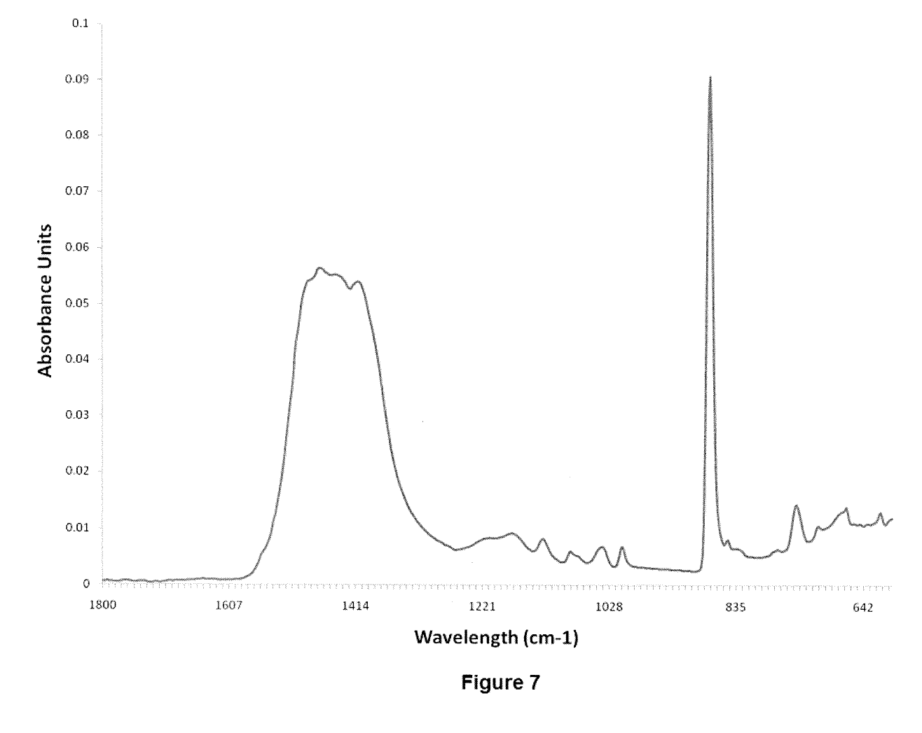

The chemical composition of calcium carbonate nanoparticles were characterized using Fourier transform infrared spectroscopy (FTIR). Samples were freeze dried overnight. FTIR spectrum (FIG. 7) of calcium carbonate nanoparticles exhibited peaks at 800 cm⁻¹ and 1400 cm⁻¹ demonstrating carbonate ion present in calcium carbonate nanoparticles.

### Example 5

### Calcium Carbonate Nanoparticle Bovine Serum Albumin Incorporation

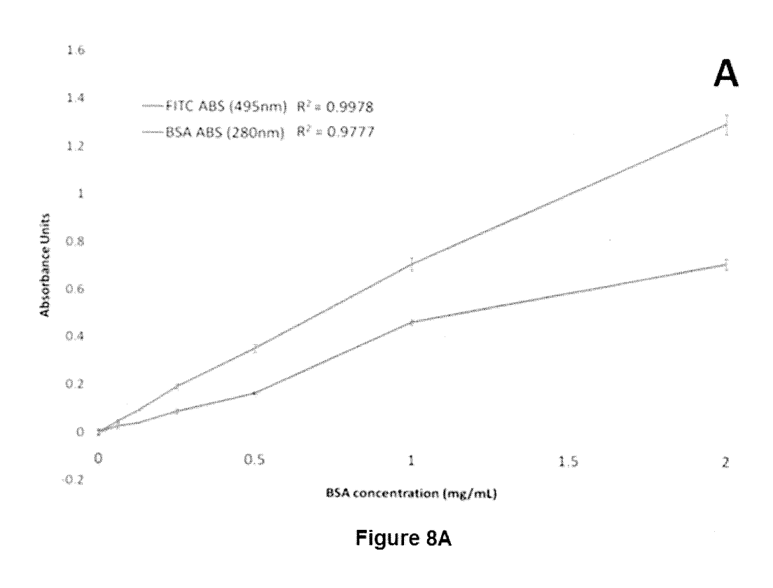

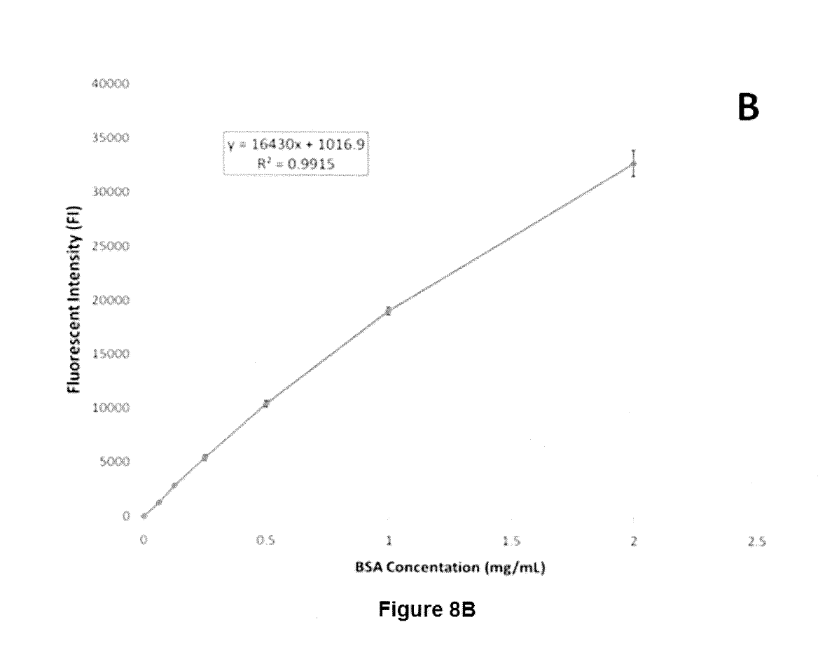

Bovine serum albumin-fluorescein isothiocyanate (BSA-FITC) conjugation was accomplished as follows. The BSA-FITC (dye:protein 5:1) conjugation was prepared by overnight incubation in 0.1M carbonate buffer, pH 9.0, and dialyzed against 0.01 M Tris-HCl, pH 7.5 (MW cutoff 10,000). BSA to FITC molar ratio was calculated using formula below. Absorbance (FIG. 8A) and fluorescent intensities (FIG. 8B) of BSA conjugated with FITC (BSA-FITC) were measured.

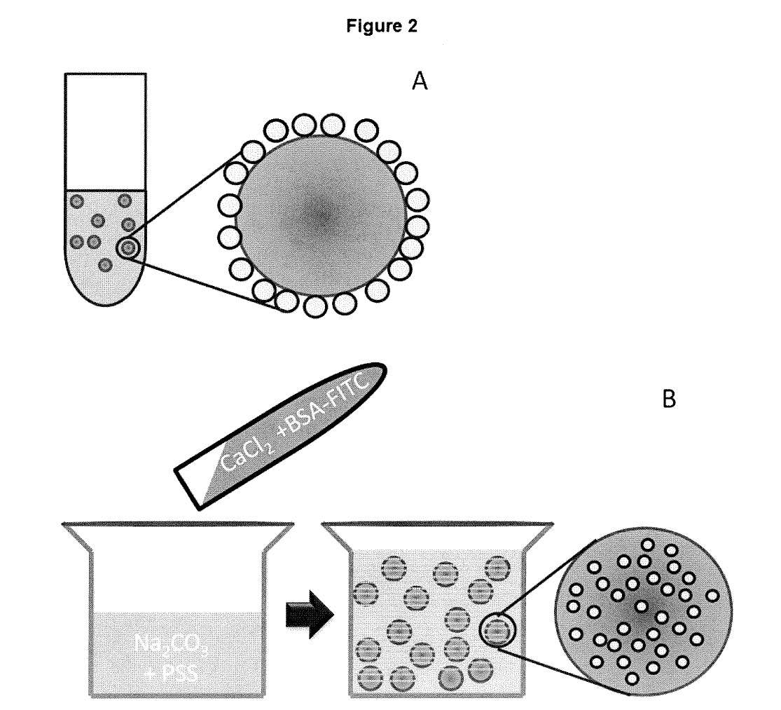

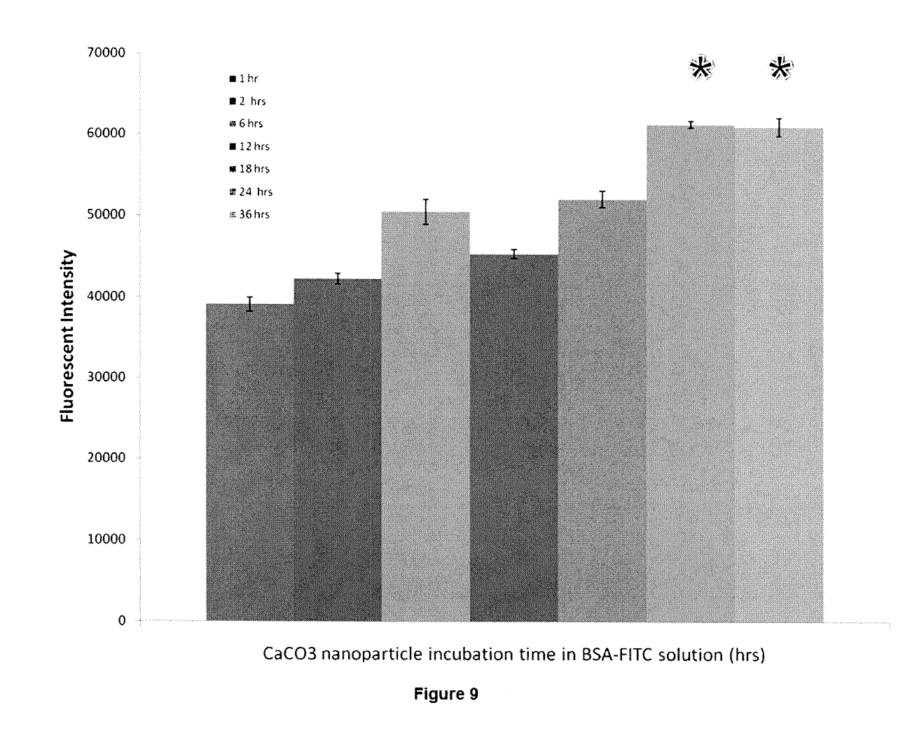

Calcium carbonate BSA-FITC incorporation was accomplished using the following methods. Two protein loading methodologies (physical adsorption and co-precipitation) were investigated for the purpose of BSA encapsulation efficiency and CNT loading capacity. A pilot study was conducted to determine an optimal incubate period for physical adsorption method. Fluorescent intensity of BSA-FITC loaded calcium carbonate nanoparticles that were incubated for different time points: 1 hr, 2 hrs, 6 hrs. 12 hrs, 18 hrs, 24 hrs, and 36 hrs. Fluorescent intensities were later measured and graphed (FIG. 9) indicating that 24 hrs or 36 hrs incubation times exhibited significantly greater fluorescent intensities when compared to incubation times less than 24 hrs (n=3, p<0.001, SEM). The first method, physical adsorption (FIG. 2A), involves incubating CNTs with FITC-BSA for 24 hours, while the second method, co-precipitation (FIG. 2B), involves adding FITC-BSA to CaCl₂ solution before mixing with NaCO₃.

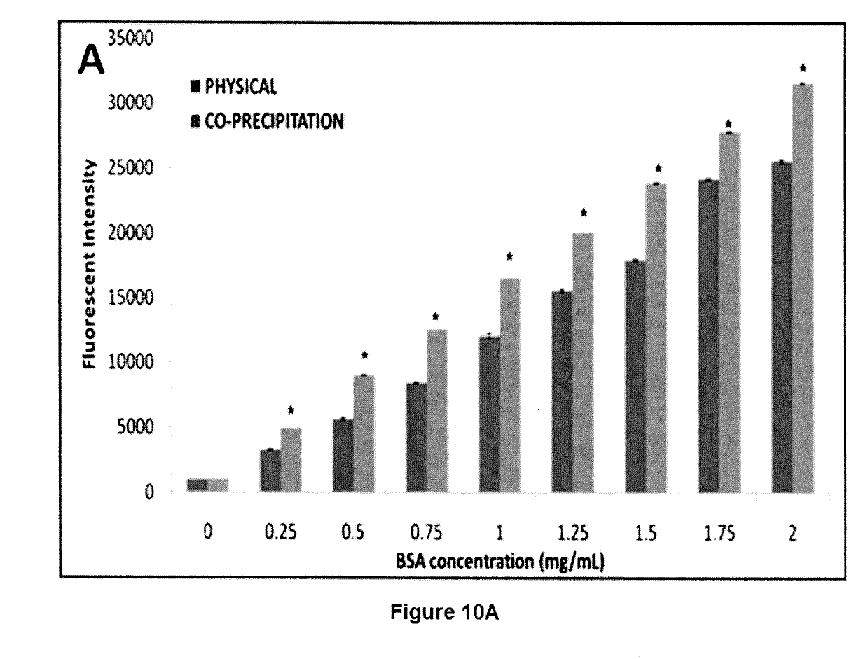

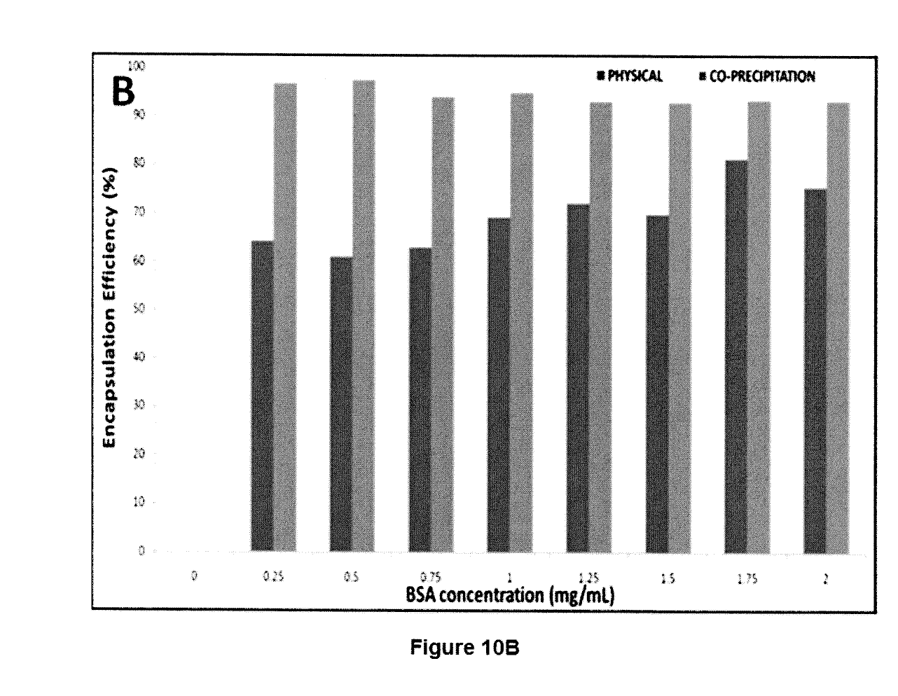

Comparison of BSA-FITC loading methods of calcium carbonate nanoparticles was made, evaluating direct physical adsorption vs. co-precipitation. Fluorescent intensities from the supernatant of both methodologies were measured using a microplate reader. The amount of BSA-FITC encapsulated, from both methods, was found by subtracting signal from reference solutions of BSA-FITC at the same concentrations. Co-precipitation method (FIG. 10A) was found to more effective than physical adsorption method. In addition maximum encapsulation efficiency (FIG. 10B) was observed at 0.50 mg/mL (97.5%) BSA-FITC concentration.

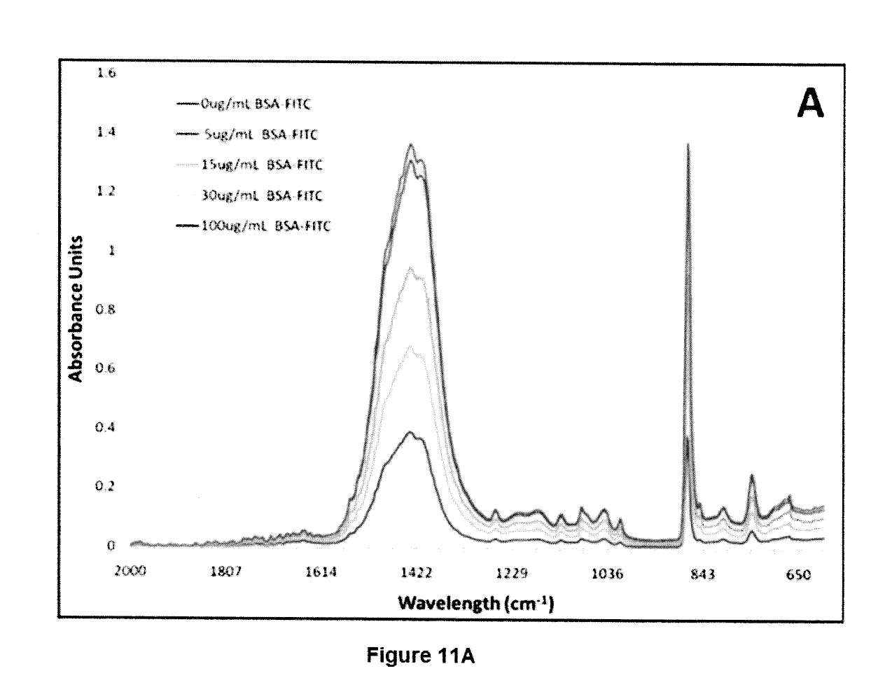

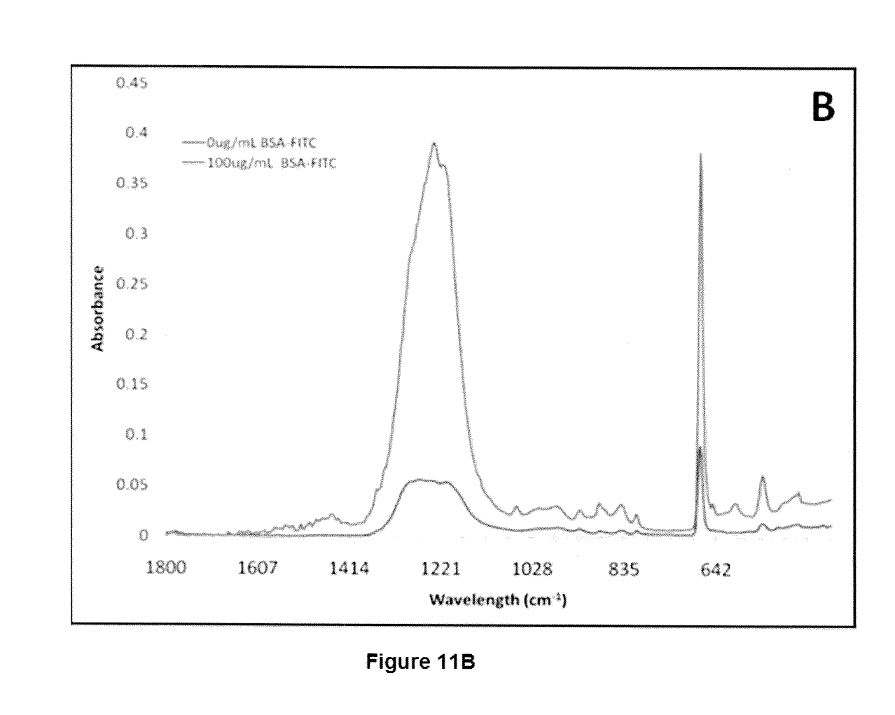

The chemical composition of bovine serum albumin (BSA) loaded calcium carbonate nanoparticles was evaluated as follows. BSA encapsulation methodologies (direct physical adsorption vs. co-precipitation) were investigated in terms of encapsulation efficiencies and co-precipitation was deemed the better method. Hence, BSA with concentrations ranging from 0 ug/mL to 100 ug/mL was loaded in calcium carbonate nanoparticles using co-precipitation. The nanoparticles were retrieved and freeze dried overnight. FTIR spectrum (FIG. 11) of BSA (0 ug/mL-100 ug/mL) loaded calcium carbonate nanoparticle exhibited peaks indicating presence of BSA since amide I region is observed at 1500-1550 cm⁻¹. In addition spectrum peaks at 800 cm⁻¹ and 1400 cm⁻¹ demonstrate carbonate ion is present in calcium carbonate nanoparticles.

### Example 6

### Effect of Polystyrene Sulfonate (PSS) on Calcium Carbonate Nanoparticles

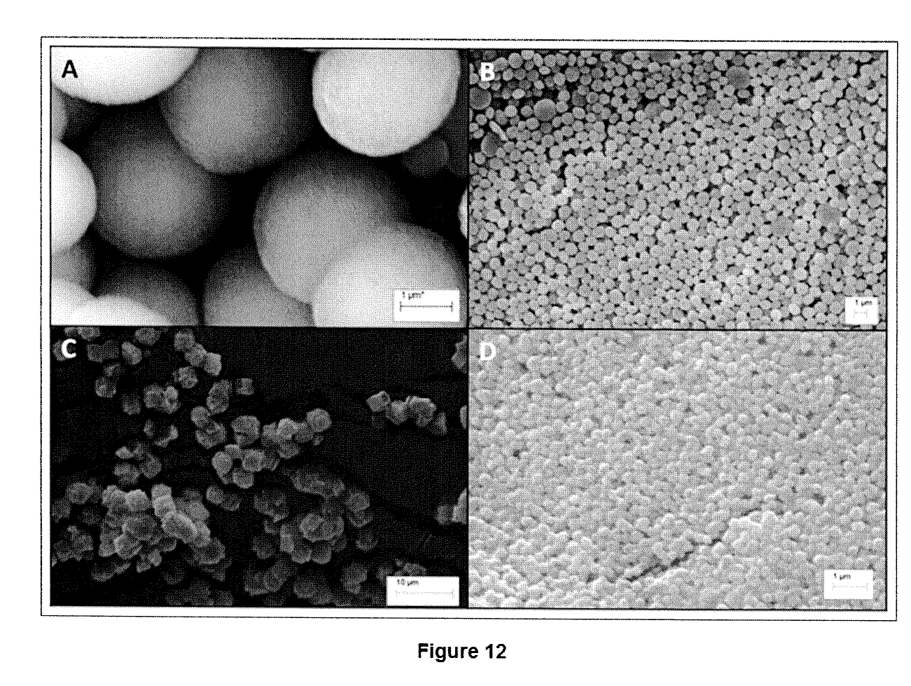

Evaluation of calcium carbonate nanoparticle morphology was conducted as follows. The purpose of this much was to investigate the effect of polystyrene sulfonate on calcium carbonate nanoparticles adding PSS (10 mg/mL) during calcium carbonate synthesis. The control for this experiment is calcium carbonate nanoparticles without any PSS added during fabrication. The nanoparticles were retrieved and nanoparticle sample preparation involved re-suspending (1 mg/mL de-ionized water, sonicating and ending with vortexing. The morphology of calcium carbonate nanoparticles was characterized using SEM where a 1.5 uL drop of suspension was placed on SEM 9 mm carbon tab and to dry under a hood. The samples were later gold/palladium coated and imaged using Ziess EVO40 SEM. Two sets of samples were prepared for each type of calcium carbonate nanoparticles: those with and without PSS. FIG. 12 itemizes SEM images taken of nanoparticles made with and without PSS.

The present disclosure shows that calcium carbonate nanoparticles can be synthesized using simple precipitation reaction between sodium carbonate (NaCO₃) and calcium chloride (CaCl₂). Calcium carbonate nanoparticles were equally sized, spherical, rough, and non-aggregated with a mean size of 315.9±1.4 nm. Zeta potential of nanoparticles were found to be −15.28±01 mV designating nanoparticles as stable and they can withstand layer-by-layer process starting with positively charged polyclectrolyte: poly(allylamine hydrochloride). Nanoparticle chemical composition was confirmed by observing carbonate ion peaks at 800 cm⁻¹ and 1400 cm⁻¹.

Two protein loading methodologies (physical adsorption and co-precipitation) were investigated for the purpose of BSA encapsulation efficiency. The first method (physical adsorption) involved incubating calcium carbonate nanoparticles with BSA-FITC for 24 hours, while the second method (co-precipitation) involved adding BSA-FITC to CaCl₂ solution before mixing with NaCO₂, and polystyrene sulfonate. The latter method was found to be significantly more effective than former method (p<0.0001, n=5, t-test). In addition, maximum encapsulation efficiency (97.5%) was observed at 0.50 mg/mL BSA-FITC concentration. Protein loaded, bovine serum albumin, calcium carbonate nanoparticles underwent further chemical analysis using FTIR where BSA amide I region peak (1500-1550 cm⁻¹) was confirmed in all BSA loaded nanoparticles and BSA amide I region peak was not seen in calcium nanoparticles sans BSA loading.

The present disclosure shows that calcium carbonate nanoparticles can be fabricated using a simple precipitation reaction between sodium carbonate (NaCO₃) and calcium chloride (CaCl₂). Equally sized, round with mean diameter of 315.9±1.4 nm and stable CNTs can be made and co-precipitation method offers best calcium carbonate nanoparticle loading. Also, these CNTs are spherical, rough, and non-aggregated rendering them appropriate LbL nanocapsule templates CNTs were negatively charged, designated as stable by zeta potential, and spherically intact demonstrating that they can withstand layer by layer (LbL) process starting with positively charged polyelectrolyte. BSA-FITC co-precipitation method 5 times more effective than physical adsorption method with maximum encapsulation efficiency (98.2%) at 0.50 mg/Ml BSA=F.

Finally, the present disclosure shows the effects of polystyrene sulfonate (PSS) on calcium carbonate nanoparticle. A strong correlation was observed between the presence of PSS and the stability of calcium carbonate nanoparticles morphology over time. Moreover, calcium carbonate nanoparticle size decreased from 1 μm-200 nm as PSS mass varied from 1-3.5 grams.

### REFERENCES CITED

The following references, to the extent that they provide exemplary procedural or other details supplementary to those set forth herein, are specifically incorporated herein by reference.

### US Patent Documents

United States Patent Publication 2007-041079
United States Patent Publication 2007-0190155
United States Patent Publication 2008-069561
United States Patent Publication 2008-0293805
United States Patent Publication 2009-0181076
United States Patent Publication 2009-0269405
United States Patent Publication 2010-0010102
United States Patent Publication 2010-134829
United States Patent Publication 2010-0266491
United States Patent Publication 2010-0303716
United States Patent Publication 2011-0123456
U.S. Pat. No. 7,090,868
U.S. Pat. No. 7,101,575
U.S. Pat. No. 7,195,780
U.S. Pat. No. 7,217,735

### Non-Patent Literature

1. cited 2010: Available from: www.cancer.org
2. Gil, P. R., et al, Nanoparticle-modified polyelectrolyte capsules. Nano Today. 30-4): p. 12-21.
3. Mariotto, A. B., et al. Projections of the Cost of Cancer Care in the United States: 2010\dot{a} [symbol] 2020. Journal of the National Cancer Institute. 103(2): p. 117-128.
4. Chiodoni, C., M. P. Colombo, and S. Sangaletti, Matricellular proteins: from homeostasis to inflammation, cancer, and metastasis. Cancer Metastasis Reviews, 2010. 29(2): p. 295-307.
5. Tanaka, T., et al., Nanotechnology for breast cancer therapy. Biomedical Microdevices, 2009. 11(1): p. 49-63.
6. Stage Inoperable IIIC, IV, Recurrent, and Metastic Breast Cancer. Breast Cancer Treatment (PDQ) 2011 Apr. 13, 2011 [cited 2011; Stage IIIB, Inoperable IIIC, IV, Recurrent, and Metastic Breast Cancer]. Available from: http://www.cancer.gov/canecrtopics/pdq/treatment/breast/healthprofessional.
7. Piccart-Gebhart, M. J., et al., Taxanes Alone or in Combination With Anthracyclines As First-Line Therapy of Patients With Metastatic Breast Cancer. Journal of Clinical Oncology, 2008. 26(12): p. 1980-1986.
8. Wilcken, N. and R. Dear, Chemotherapy in metastatic breast cancer: A summary of all randomised trials reported 2000-2007. European Journal of Cancer (Oxford, England: 1990), 2008. 44(15): p. 2218-2225.
9. Taxanes. 2011 [cited 2011; Taxanes]. Available from: http://toxipedia.org/display/toxipedia/Taxanes.
10. Scanio, M. and R. J. Frey, Paclitaxel. Vol. 2. 2006, Detroit: Gale.
11. Paclitaxel. Paclitaxel 2009 2009 [cited 2011; Paclitaxel]. Available from: http://www.paclitaxel.org/.
12. Vachani, C. Paclitaxel (Taxol). OncoLink 2007 Jul. 1, 2007 [cited 2011; Paclitaxel (Taxol)]. Available from: http://oncolink.org/treatment/article.cfm?e=2&s=10&id=145.
13. Paclitaxel. Vol. 5. 2006, Amsterdam: Elsevier.
14. Gelderblom, H., et al., Cremophor EL: the drawbacks and advantages of vehicle selection for drug formulation. European Journal of Cancer, 2001. 37(13): p. 1590-1598.
15. Yezhelyev, M. V., et al., Emerging use o nanoparticles in diagnosis and treatment of breast cancer. The Lancet Oncology, 2006. 7(8): p. 657-667.
16. Brouwer. E., et al., Measurement of Fraction Unbound Paclitaxel in Human Plasma. Drug Metabolism and Disposition, 2000. 28(10): p. 1141-1145.
17. Ferrari, M., Frontiers in cancer nanomedicine: directing mass transport through biological barriers. Trends in Biotechnology, 2010. 28(4): p. 181-188.
18. Malam, Y., M. Loizidou, and A. M. Seifalian, Liposomes and nanoparticles: nanosized vehicles for drug delivery in cancer. Trends in Pharmacological Sciences, 2009. 30(11): p. 592-599.
19. Ferrari, M., Cancer nanotechnology; opportunities and challenges. Nature Reviews. Cancer, 2005. 5(3): p. 161-171.
20, Li, K. C. P., et al. Molecular imaging applications in nanomedicine. Biomedical Microdevices, 2004. 6(2): p. 113-116.
21. Gajria, D., A. Seidman, and C. Dang, Adjuvant Taxanes: More to the Story. Clinical Breast Cancer. 10(0): p. S41-S49-S41-S49.
22, Gradishar, W. J., et al., Phase III Trial of Nanoparticle Albumin-Bound Paclitaxel Compared With Polyethylated Castor Oil A-Based Paclitaxel in Women With Breast Cancer. Journal of Clinical Oncology, 2005. 23(31): p. 7794-7803.
23. Kratz., F., Albumin as a drug carrier: design of prodrugs, drug conjugates and nanoparticles. Journal of Controlled Release: Official Journal of the Controlled Release Society, 2008. 132(3): p. 171-183.
24. Quinlan, G. J., G. S. Martin, and T. W. Evans, Albumin: Biochemical properties and therapeutic potential. Hepatology, 2005. 41(6): p. 1211-1219.
25. Farrugia, A., Albumin usage in clinical medicine: tradition or therapeutic?Transfusion Medicine Reviews. 24(1): p. 53-63.
26. Shu, S., et al., Hollow and degradable polyelectrolyte nanocapsules for protein drug delivery. Acta Biomater, 2010. 6(1): p. 210-7.
27. De Geest, B. G., et al., Release mechanisms for polyelectrolyte capsules. Chemical Society Reviews, 2007. 36(4): p. 636-636.
28. Johnston, A. P. R., et al., Layer-by-layer engineered capsules and their applications. Current Opinion in Colloid & Interface Science, 2006. 11(4): p. 203-209.
29. Becker, A. L., A. P. R. Johnston, and F. Caruso, Layer-by-layer-assembled capsules and films for therapeutic delivery. Small (Weinheim an Der Bergstrasse, Germany), 2010. 6(17): p. 1836-1852.
30. Gil, P. R., et al., Nonoparticle-modified polyelectrolyte capsules. Nano Today. 3(3-4): p. 12-21.
31. De Geest, B. G., G. B. Sukhorukov, and H. Möhwald, The pros and cons of polyelectrolyte capsules in drug delivery. Expert Opinion on Drug Delivery, 2009. 6(6): p. 613-624.
32. Calcium Carbonate. 2007, Westport, Conn.: Greenwood Press.
33. Shan, D., et al., Direct electrochemistry and electrocatalysis of hemoglobin entrapped in composite matrix based on chitosan and CaCO3 nanoparticles. Electrochemistry Communications, 2007. 9(4): p. 529-534.
34. Calcium carbonate. Vol. 1. 2008, Detroit: Gale.
35. Cölfen, H., Precipitation of carbonates: recent progress in controlled production of complex shapes. Current Opinion in Colloid & Interface Science, 2003. 8(1): p. 23-31.
36. Shen, Q., et al., Properties of Amorphous Calcium Carbonate and the Template Action of Vaterite Spheres. The Journal of Physical Chemistry B, 2006. 110(7): p. 2994-3000.
37. Wei, H., et al., Effect of ionic surfactant-polymer complexes on the crystallization of calcium carbonate. Journal of Crystal Growth, 2004. 264(1-3): p. 424-429.
38. Rivera-Gil, P., et al., Intracellular Processing of Proteins Mediated by Biodegradable Polyelectrolyte Capsules. Nano Letters, 2009. 9(12): p. 4398-4402.
39. Decher, G. and J. Schmitt, Fine-Tuning of the film thickness of ultrathin multilayer films composed of consecutively alternating layers of anionic and cationic polyelectrolytes, in Trends in Colloid and Interface Science VI. 1992, Steinkopff: Darmstadt, p. 160-164,
40. Polyelectrolytes. 2008, Hoboken, N.J.: Wiley-Interscience.
41. Chang, C. and Z. Werb, The many faces of metalloproteases: cell growth, invasion, angiogenesis and metastasis. Trends in Cell Biology, 2001. 11(11): p. S37-43-S37-43,
42. Ehrenfeld, P., et al., Activation of kinin B1 receptor increases the release of metalloproteases-2 and -9 from both estrogen-sensitive and -insensitive breast cancer cells. Cancer Letters, 2011. 301(1): p. 106-118.
43. Kyriakides, T. R., et al., Mice that lack matrix metalloproteinase-9 display delayed wound healing associated with delayed reepithelization and disordered collagen fibrillogenesis. Matrix Biology: Journal of the International Society for Matrix Biology, 2009. 28(2): p. 65-73.
44. Westermarck, J. and V.-M. KÄHÄRi, Regulation of matrix metalloproteinase expression in tumor invasion. The FASEB Journal, 1999. 13(8): p. 781-792.
45. Sternlicht, M. D. and Z. Werb, How matrix metalloproteinases regulate cell behavior. Annual Review of Cell and Developmental Biology, 2001. 17: p. 463-516.
46. Visse, R. and H. Nagase, Matrix Metalloproteinases and Tissue Inhibitors of Metalloproteinases: Structure, Function, and Biochemistry. Circ Res, 2003. 92(8): p. 827-839.
47. Nöel, A., M. Jost, and E. Maquoi, Matrix metalloproteinases at cancer tumor-host interface. Seminars in Cell & Developmental Biology, 2008. 19(1): p. 52-60.
48. Egeblad, M. and Z. Werb, New functions for the matrix metalloproteinases in cancer progression. Nat Rev Cancer, 2002. 2(3): p. 161-174.
49. Chatterjee, S. K. and B. R. Zetter, Cancer biomarkers: knowing the present and predicting the future. Future Oncology (London, England), 2005. 1(1): p. 37-50.
50. Sarkar, N., et al., Matrix metalloproteinase-assisted triggered release of liposomal contents. Bioconjugate Chemistry, 2008. 19(1): p. 57-64.
51. Roth, C. H., Fundamentals of logic design. 5th ed. 2004, Belmont, Calif.: Thomson/Brooks/Cole, xxi, 687 p.
52. Benenson, Y., et al., Programmable and autonomous computing machine made of biomolecules. Nature, 2001. 414(6862): p. 430-4.
53. Benenson, Y., et al., An autonomous molecular compute or logical control of gene expression. Nature, 2004. 429(6990): p. 423-9.
54. Balzani, V., A. Credi, and M. Venturi, Molecular logic circuits. Chemphyschem, 2003. 4(1): p. 49-59.
55. Shapiro, E. and Y. Benenson, Bringing DNA Computers To Life. Scientific American, 2006. 294(5): p. 44-51.
56. Yao, G., et al., FloDots: luminescent nanoparticles. Analytical and Bioanalytical Chemistry, 2006.
57. Jin, Y., et al., Silica Nanoparticles with Continuously Tunable Sizes: Synthesis and Size Effects on Cellular Contrast Imaging. Chemistry of Materials, 2008. 20(13): p. 4411-4419,
58. Wang, L., W. Zhao, and W. Tan, Bioconjugated silica nanoparticles: Development and applications. Nano Research, 2008. 1(2): p. 99-115.
59. De Geest, B. G., G. B. Sukhorukoy, and H. Möhwald, The pros and cons Of polyelectrolyte capsules in drug delivery. Expert Opinion on Drug Delivery, 2009. 6: p. 613-624.
60. Wang, C., et al., Combination of adsorption by porous CaCO3 microparticles and encapsulation by polyelectrolyte multilayer films for sustained drug delivery. Int J Pharm, 2006. 308(1-2): p. 160-7.
61. Rauscher, F., P. Veit, and K. Sundmacher, Analysis of a technical-grade w/o-microemulsion and its application for the precipitation of calcium carbonate nanoparticles. Colloids and Surfaces A: Physicochemical and Engineering Aspects, 2005. 254(1-3): p. 183-191.
62. Volodkin, D. V., et al., Matrix polyelectrolyte microcapsules: new system far macromolecule encapsulation. Langmuir, 2004. 20(8): p. 3398-406.
63. Petrov, A. I., D. V. Volodkin, and G. B. Sukhorukov, Protein-calcium carbonate coprecipitation: a tool for protein encapsulation. Biotechnol Prog, 2005. 21(3): p. 918-25.
64. Cai, A., et al., Direct Synthesis of Hollow Vaterite Nanospheres from Amorphous Calcium Carbonate Nanoparticles via Phase Transformation. The Journal of Physical Chemistry C, 2008. 112(30): p. 11324-11330.
65. Bawarski, W. E., et al., Emerging nanopharmaceuticals. Nanomedicine: Nanotechnology, Biology and Medicine, 2008. 4(4): p. 273-282.
66. Agarwal, A., et al., Stable nanocolloids of poorly soluble drugs with high drug content prepared using the combination of sonication and layer-by-layer technology. Journal of Controlled Release, 2008. 128(3): p. 255-260.
67. Kroemer, G. et al., Classification of cell death: recommendations of the Nomenclature Committee on Cell Death. Cell Death & Differentiation, 2005. 12: p. 1463-1467.
68. Thornberry, N. A. and Y. Lazebnik, Caspases: enemies within. Science (New York, N.Y.), 1998. 281(5381): p. 1312-1316.
69. Anandhakumar, S., V. Nagaraja, and A. M. Raichur, Reversible polyelectrolyte capsules as carriers for protein delivery. Colloids and Surfaces B: Biointerfaces, 2010. 78(2): p. 266-274.
70. Sukhorukov, G. B., et al., Porous calcium carbonate microparticles as templates for encapsulation of bioactive compounds. Journal of Materials Chemistry, 2004. 14(14): p. 2073-2073.
71. Tong, W., et al., Charge-Controlled Permeability of Polyelectrolyte Microcapsules. The Journal of Physical Chemistry B, 2005. 109(27): p. 13159-13165.
72. Shu, S., et al., Hollow and degradable polyelectrolyte nanocapsules far protein drug delivery. Acta Biomaterialia, 2010. 6(1): p. 210-217.
73. Möhwald, H., From Langmuir monolayers to nanocapsules. Colloids and Surfaces A: Physicochemical and Engineering Aspects, 2000. 171(1-3): p. 25-31.
74. Schwiertz, J, et al., Calcium phosphate nanoparticles as templates for nanocapsules prepared by the layer-by-layer technique. Journal of Materials Chemistry, 2008. 18(32): p. 3831-3831.
75. von Maltzahn, G., et al., Nanoparticle self-assembly gated by logical proteolytic triggers. J Am Chem Soc, 2007. 129(19): p. 6064-5.
76. Sarkar, N. R., et al., “Uncorking” of liposomes by matrix metalloproteinase-9. Chemical Communications (Cambridge, England), 2005(8): p. 999-1001.
77. Antipov, A. A. and G. B. Sukhorukov, Polyelectrolyte multilayer capsules as vehicles with tunable permeability. Advances in Colloid and Interface Science, 2004. 111(1-2): p. 49-61.
78. Bremer, C., C.-H. Tung, and R. Weissleder, In vivo molecular target assessment matrix metalloproteinase inhibition. Nat Med, 2001. 7(6): p. 743-748.
79. Turk, B. E., et al., Determination of protease cleavage site motifs using mixture-based oriented peptide libraries. Nat Biotechnol, 2001. 19(7): p. 661-667.
80. Chluba, J., et al., Peptide Hormone Covalently Bound to Polyelectrolytes and Embedded into Multilayer Architectures Conserving Full Biological Activity. Biomacromolecules, 2001. 2(3): p. 800-805.
81. Tsai, W.-B., et al., Polyelectrolyte multilayer films functionalized with peptides for promoting osteoblast functions. Acta Biomaterialia, 2009. 5(9): p. 3467-3477.
82. Seliktar, D., et al., MMP-2 sensitive, VEGF-bearing bioactive hydrogels for promotion of vascular healing. Journal of Biomedical Materials Research Part A, 2004. 68(4): p. 704-716.
83. Petrov, A. I., D. V. Volodkin, and G. B. Sukhorukov, Protein—Calcium Carbonate Coprecipitation: A Tool for Protein Encapsulation. Biotechnology Progress, 2005. 21(3): p. 918-925.

What is claimed:

1. A nanocapsule composition comprising:
a calm carbonate core:
a bilayer comprising polystyrene sulfonate and poly(allylamine hydrochloride), wherein the bilayer substantially surrounds the calcium carbonate core; and
a substrate capable of being activated b) contacting a biomarker for a disease state, wherein the substrate is immobilized onto the surface of the bilayer.

2. The nanocapsule composition of claim 1, further comprising additional polystyrene sulfonate and poly(allylamine hydrochloride) bilayers, wherein the bilayers substantially surround the calcium carbonate core.

3. The nanocapsule composition of claim 1, wherein the substrate is an MMP-cleavable substrate.

4. The nanocapsule composition of claim 3, wherein the MMP-cleavable substrate is MMP-2-cleavable, MMP-9-cleavable.

5. The nanocapsule composition of claim 1, wherein the substrate is a combination of both MMP-2 cleavable substrate and MMP-9-cleavable substrate.

6. A method for treating a disease state, comprising the step of:
administering the nanocapsule composition claim 1 to an animal.
